(19) United States
(12) Patent Application Publication
(10) Pub. No.: US 2006/0134144 A1
(43) Pub. Date: Jun. 22, 2006

(54) Mucoadhesive composition and formulation for solubilization of insoluble drugs and preparation method thereof

(76) Inventors: Hesson Chung, Incheon (KR); Seo-Young Jeong, Gyeonggi-Do (KR); Ick-Chan Kwon, Seoul (KR); Yeong-Taek Park, Gyeonggi-Do (KR); In-Hyun Lee, Seoul (KR)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/521,989

(22) PCT Filed: Jul. 21, 2003
(86) PCT No.: PCT/KR03/01443

§ 371 Date: Nov. 3, 2005

(30) Foreign Application Priority Data
Jul. 20, 2002 (KR) 10-2002-0042793

(51) Int. Cl. A61K 9/00; A61K 31/225

(52) U.S. Cl. 424/400; 514/547

(57) ABSTRACT

The present invention relates to a novel mucoadhesive composition for solubilization of insoluble drugs; its formulation including pharmaceutical compounds; and the preparation methods thereof wherein said solubilizing composition comprises 4˜90% by weight of at least one selected from the monoglycerides and 0.01˜90% by weight of at least one oil. The present invention relates to a novel mucoadhesive composition including pharmaceutical compounds; and the preparation methods thereof wherein said solubilizing composition including emulsifiers is composed of 4˜90% by weight of at least one selected from the monoglycerides, 0.01˜90% by weight, of at least one oil, and 0.01˜90% by weight of at least one selected from the emulsifiers. The compositions of the present invention is suitable as drug delivery systems since they exist as mucoadhesive liquid at physiological temperatures even though they exist as liquid or semi-solid at room temperature.

### TECHNICAL FIELD

The present invention relates to a novel mucoadhesive composition for solubilization of insoluble drugs; its formulation including pharmaceutical compounds; and the preparation methods thereof, wherein said solubilizing composition is composed of 4˜90% by weight of at least one selected from the monoglycerides and 0.01˜90% by weight of at least one oil. The present invention also relates to a novel mucoadhesive composition including emulsifiers for solubilization of insoluble drugs; its formulation including pharmaceutical compounds; and the preparation methods thereof wherein said solubilizing composition including emulsifiers is composed of 4˜90% by weight of at least one selected from the monoglycerides, 0.01˜90% by weight of at least one oil, and 0.01˜90% by weight of at least one selected from the emulsifiers. The compositions of the present invention are suitable as drug delivery systems since they exist as mucoadhesive liquid at physiological temperatures even though they exist as liquid or semi-solid at room temperature.

### BACKGROUND ART

Solubilization process is a very important step in preparing the delivery systems of insoluble drugs. To solubilize insoluble drugs, a variety of compositions including fats, lipids and oils have been prepared in the past. These compositions, however, are decomposed by lipase in the intestine or solubilized by bile salts to form mixed micelles resulting in lowered absorption of the encapsulated drugs. To overcome these problems and to increase the absorption rate, nano-sized lipid particles have also been prepared by the aid of emulsifiers since the particles of small size were absorbed through the intestinal cells easily. The present inventors have found, however, that the oily compositions that are mucoadhesive and can dissolve insoluble drug can help increase the absorption rate of the encapsulated drug when taken orally even if the oily composition cannot be dispersed in water homogeneously.

Since mucoadhesive drug delivery systems can be adsorbed on the intestinal absorptive cells via oral, buccal or intranasal administration and slowly release the encapsulated drugs in the vicinity of the site of absorption, drug absorption rate can be increased when taken orally or applied directly on the wound.

Conventionally used mucoadhesive drug delivery systems are mainly polymeric materials including DEAE dextran, polycarbophil, sodium alginate, hydroxypropyl methylcellulose (HPMC) and Carbopol 934 (BF Goodrich, USA). Among lipids, monoglycerides are known to have high mucoadhesiveness. The mucoadhesiveness of the monoglycerides is the highest when they exist as the precursors of cubic or hexagonal phases. Among these precursors, Elyzol gel for the treatment of periodontal disease comprising metronidazole benzoate, monoglyceride and unsaturated triglycerides with a small amount of water (20%) is commercially available [Norling et. al., Formulation of a drug delivery system based on a mixture of monoglycerides and triglycerides for use in the treatment of periodontal disease (1992) J. Clin. Periodontol. vol. 19, page 687-692]. Mucoadhesive hexagonal liquid crystalline phase is formed inside the periodontal pocket when Elyzol is injected.

The precursors, however, become mucoadhesive only when they come in contact with mucosal cells. If the precursors meet intestinal fluid and become hexagonal or cubic phase before contacting mucosal cells, they lose the mucoadhesiveness to a great extent. Even if the precursor reaches the mucosal cells before absorbing intestinal fluid, they can be degraded by the intestinal enzymes. Also, the formed cubic or hexagonal phase covers only limited areas of the intestine, drug is absorbed at the site of attachment only. This problem arises since the cubic phase that monoglyceride and water forms has a very high viscosity and does not migrate to lower parts of the intestine. The composition of the present invention, however, contains oils of low viscosity that helps the composition to flow inside the intestine and to coat the interior of intestine. Therefore, the amount of drug absorption per unit contact area between mucosal cells and the composition increases when the whole intestine is considered.

The drug content also increases since the oils with low viscosity and high solubility for lipophilic drugs are included in the composition. For example, the solubility of pyrene, a model drug, in tricaprylin, a saturated triglyceride, is 92.9 mg/ml, whereas that in monoolein is 43.6 mg/ml. By adding tricaprylin in the composition, more pyrene can be solubilized. Since the composition of the present invention does not contain water, the composition is stable for a long period of time without undergoing oxidation and hydrolysis of the components.

Monoolein is mucoadhesive when it exists as a low viscous liquid. The melting point of monoolein of high purity (99.5% pure) is 37° C. and that of Myverol 18-99 (Danisco, Denmark) is 35˜40° C. The melting point of monoolein is similar to the body temperature, and it can absorb the stomach fluid or intestinal fluid if the soft capsule containing monoolein dissolves in stomach or intestine, respectively. Since the cubic phase that monoolein forms upon absorbing water is a highly viscous gel at 37° C., it only coats a limited contact area. The present invention, on the other hand, forms a low viscousity phase that can coat the intestinal cells evenly providing a wider area for drug absorption.

If the drug is encapsulated in the oil without monoglyceride, the composition is adsorbed on the intestine momentarily, but the drug absorption is ineffective since the oil has to be digested before being absorbed. Monoglyceride, however, can be absorbed directly without being digested on the mucosal cells and therefore can carry the drug with it. Therefore, the composition of the present invention containing a mixture of monoglyceride and oil can coat a wide surface area of intestine, can load drug at a high concentration and can help absorption of the drug without being digested.

The single phase oily composition made of monoglyceride and oil or the composition of monoglyceride, oil and emulsifier have not been used as oral or buccal drug delivery systems in the past. Compositions including oils and water have been used for oral delivery. In these cases, the compositions form an L2 phase, in which small water droplets are formed inside the oil phase. The drugs are loaded inside and released from the water droplets. These compositions in L2 phase have many shortcomings when compared to the compositions of the present invention. Once water is introduced into the system, the components can become destabilized due to oxidation and/or hydrolysis. Also the insoluble drugs can precipitate out with time. Also the administration dose would increase as the amount of added water increases.

When the composition of the present invention includes an emulsifier, the emulsifier can help the composition to be dispersed inside the intestine into microparticles with the diameter of a few micrometers since the movement of intestine would help micronization process. Therefore, it is possible to coat as wide area of the intestinal wall in case the composition includes an emulsifier.

The present inventors have proven that the composition containing monoglycerides and oils can solubilize the insoluble drugs, help preventing the precipitation of the drugs, and can be dispersed into microparticles in water, can be adsorbed into the intestinal wall and therefore can increase oral bioavailability when orally consumed.

### SUMMARY OF THE INVENTION

The object of the present invention is to provide a composition for solubilization of insoluble drugs and the preparation method thereof. Another object of the present invention is to provide a formulation by adding drugs in the above composition for solubilization of insoluble drugs to be used as drug delivery systems and the preparation method thereof.

### DETAILED DESCRIPTION OF THE INVENTION

The present invention relates to a solubilizing composition of homogeneous oily mixture comprising monoglyceride and oil for solubilization of insoluble drugs, and the preparation method thereof.

Also, the present invention relates to a novel formulation comprising the above solubilizing composition and pharmaceutical compounds, and the preparation method thereof.

Also the present invention relates to a solubilizing composition including emulsifiers for solubilization of insoluble drugs comprising the above solubilizing composition and emulsifiers.

And the present invention also relates to another novel formulation comprising the above solubilizing composition including emulsifier and pharmaceutical compounds, and the preparation method thereof.

In what follows, the present invention will be described in detail.

The present invention relates to a mucoadhesive composition for solubilization of insoluble drugs.

Specifically, the above composition is composed of 4˜90% by weight of at least one selected from the monoglycerides and 0.01˜90% by weight of at least one oil (with respect to the total weight of the composition).

The above composition can be prepared by mixing at least one monoglyceride and at least one oil at room or elevated temperatures.

The above monoglycerides are selected from a group consisting of one or more saturated or unsaturated monoglycerides having 10˜22 carbon atoms in the hydrocarbon chain. Monoglycerides is selected preferably from a group consisting of monoolein, monopalmitolein, monomyristolein, monoelaidin and monoerucin and from a group consisting of the mixture of monoglycerides semi-synthesized from triglycerides of vegetable or animal oil, and more preferably monoolein.

The above oil is selected preferably from a group consisting of triglycerides, iodinated oil and vegetable or animal oil.

The above triglycerides are selected from a group consisting of one or more saturated or unsaturated triglycerides having 2˜20 carbon atoms in the hydrocarbon chain. For instance, triacetin, tributyrin, tricaproin, tricaprylin, tricaprin or triolein can be used.

The above iodized oils include iodized poppy seed oil such as Lipiodol, Ethiodol and iodized soybean oil.

The above vegetable oils include soybean oil, cottonseed oil, olive oil, poppyseed oil, linseed oil and sesame oil.

The above animal oils include squalane and squalene.

Also the above composition can additionally include other additives up to 5% by weight. For instance, the composition can further comprise alcohol, polyol or Cremophor to improve the solubility of the insoluble drugs, tocopherol or tocopherol acetate to prevent oxidation, and fatty acid, fatty acid ester or fatty acid alcohol to increase drug absorption.

The above solubilizing composition can be prepared by mixing 4˜90% by weight of at least one selected from the monoglycerides and 0.01˜90% by weight of at least one oil at temperatures lower than 50° C. to obtain a homogeneous mixture. The monoglycerides and oils used in preparing the solubilizing composition are the same as described above.

The preparation method described above is only one of many possible methods, and other preparation method can also be used to obtain the above composition.

Also the present invention provides mucoadhesive composition including emulsifiers for solubilization of insoluble drugs More particularly, the above composition is composed of 4˜90% by weight of at least one selected from the monoglycerides, 0.01˜90% by weight, of at least one oil and 0.01˜90% by weight of at least one emulsifier (with respect to the total weight of the composition).

The above composition can be prepared by adding at least one monoglyceride, at least one oil and at least one emulsifier at room or elevated temperatures.

The above monoglycerides are selected from a group consisting of one or more saturated or an unsaturated monoglycerides having 10˜22 carbon atoms in the hydrocarbon chain. Monoglycerides is selected preferably from a group consisting of monoolein, monopalmitolein, monomyristolein, monoelaidin and monoerucin and from a group consisting of the mixture of monoglycerides semi-synthesized from triglycerides of vegetable or animal oil, and more preferably monoolein.

The above oil is selected preferably from a group consisting of triglycerides, iodinated oil and vegetable or animal oil that can solubilize insoluble drugs.

The above triglycerides are selected from a group consisting of one or more saturated or unsaturated triglycerides having 2˜20 carbon atoms in the hydrocarbon chain. For instance, triacetin, tributyrin, tricaproin, tricaprylin, tricaprin or triolein can be used.

The above iodized oils include iodized poppy seed oil such as Lipiodol, Ethiodol and iodized soybean oil.

The above vegetable oils include soybean oil, cottonseed oil, olive oil, poppyseed oil, linseed oil and sesame oil.

The above animal oils include squalane and squalene.

The emulsifier is selected from the group consisting of a phospholipid, a non-ionic surfactant, an anionic surfactant, a cationic surfactant, and a bile acid.

The phospholipid is selected from the group consisting of a phosphatidylcholine (PC) and its derivative, a phosphatidylethanolamine (PE) and its derivative, a phosphatidylserine (PS) and its derivative or a polymeric lipid wherein a hydrophilic polymer is conjugated to the lipid headgroup.

The non-ionic surfactant is selected from the group consisting of a poloxamer (also known as Pluronic: polyoxyethylene-polyoxypropylene copolymer), a sorbitan ester (Span), a polyoxyethylene sorbitan (Tween) or a polyoxyethylene ether (Brij).

The anionic surfactant is selected from the group consisting of a phosphatidylserine (PS) and its derivative, a phosphatidic acid (PA) and its derivative or sodium dodecyl sulfate (SDS).

The cationic surfactant is selected from the group consisting of 1,2-dioleyl-3-trimethylammonium propane (DOTAP), dimethyldioctadecylammonium bromide (DDAB), N-[1-(1,2-dioleyloxy)propyl]-N,N,N-trimethylammonium chloride (DOTMA), 1,2-dioleyl-3-ethylphosphocholine (DOEPC) and 3β-[N-[(N′,N′-dimethylamino)ethan]carbamoyl]cholesterol (DC-Chol).

The bile acid is selected from the group consisting of cholic acid, its salt and derivatives; deoxycholic acid, its salt and derivatives; chenocholic acid, its salt and derivatives; and lithocholic acid, its salt and derivatives.

Other additives can be added to the above composition including emulsifiers to be within 5% by weight. And the examples are fatty acids, fatty acid esters and fatty acid alcohols (with respect to the total weight of the composition). For instance, the composition can further comprise alcohol, polyol or Cremophor to improve the solubility of the insoluble drugs, tocopherol or tocopherol acetate to prevent oxidation, and fatty acid, fatty acid ester or fatty acid alcohol to increase drug absorption.

The above solubilizing composition including emulsifiers can be prepared by mixing 4˜90% by weight of at least one selected from the monoglycerides, 0.01˜90% by weight of at least one oil and 0.01˜90% by weight of at least one emulsifier at temperatures lower than 50° C. to obtain a homogeneous viscous mixture. The monoglycerides, oils and emulsifiers used in preparing the solubilizing composition are the same as described above.

The preparation method described above is only one of many possible methods, and other preparation method can also be used to obtain the above composition including emulsifiers.

The compositions for solubilization of insoluble drugs with or without emulsifiers according to the present invention can be administered via various routes including oral administration, buccal administration, mucosal administration, intranasal administration, intraperitoneal administration, subcutaneous injection, intramuscular injection, transdermal administration, intratumoral administration, and more preferably an oral administration.

The compositions for solubilization of insoluble drug of the present invention exist as gel or in semi-solid form depending on the composition at room temperature. Also the compositions of the present invention are stable for a long period of time since the physical property of the composition does not change and the components do not degrade with time. Also the compositions for solubilization of insoluble drug of the present invention can be easily dispersed in water or in aqueous solutions to produce particles bigger than 500 nm in diameter, and the absorbance of the dispersion at 400 nm is higher than 0.35 (preferably 1˜4). Since the above dispersion of the compositions for solubilization of insoluble drug of the present invention does not form precipitation of the drug upon a long-time storage, the compositions of the present invention are efficient in solubilizing the insoluble drugs. Since the compositions of the present invention are highly mucoadhesive in the intestine, they adhere onto absorptive cells in the intestine wherein the drug can be absorbed directly into the cells. The viscosity of the compositions is high enough (approximately 60˜200 centipoises) to be adsorbed on a large area of the intestinal wall, thereby increasing the amount of drug absorption per unit area. Another factor that helps increasing the bioavailability of drug in the composition for solubilization of insoluble drug of the present invention is that it is composed of monoglycerides, which can be absorbed into the intestinal cells without being digested.

The present invention provides mucoadhesive formulations for solubilization of insoluble drugs that can be used as drug delivery systems.

Specifically, the above formulation is composed of 4˜90% by weight of at least one selected from the monoglycerides, 0.01˜90% by weight of at least one oil and 0.01˜20% by weight of insoluble drug (with respect to the total weight of the composition).

The above formulation can be prepared by mixing at least one monoglyceride, at least one oil and insoluble drug at room or elevated temperature.

The above monoglycerides are selected from a group consisting of one or more saturated or unsaturated monoglycerides having 10˜22 carbon atoms in the hydrocarbon chain. Monoglyceride is selected preferably from a group consisting of monoolein, monopalmitolein, monomyristolein, monoelaidin and monoerucin and from a group consisting of the mixture of monoglycerides semi-synthesized from triglycerides of vegetable or animal oil, and more preferably monoolein.

The above oil is selected preferably from a group consisting of triglycerides, iodinated oil and vegetable or animal oil.

The above triglycerides are selected from a group consisting of one or more saturated or unsaturated triglycerides having 2˜20 carbon atoms in the hydrocarbon chain. For instance, triacetin, tributyrin, tricaproin, tricaprylin, tricaprin or triolein can be used.

The above iodized oils include iodized poppy seed oil such as Lipiodol, Ethiodol and iodized soybean oil.

The above vegetable oils include soybean oil, cottonseed oil, olive oil, poppyseed oil, linseed oil and sesame oil.

The above animal oils include squalane and squalene.

Examples of the insoluble drugs that can be used in the present invention are antivirals, steroidal anti-inflammatory drugs (SAID), non-steroidal anti-inflammatory drugs (NSAID), antibiotics, antifungals, vitamins, hormones, retinoic acid, prostaglandins, prostacyclins, anticancer drugs, antimetabolitic drugs, miotics, cholinergics, adrenergic antagonists, anticonvulsants, antianxiety agents, major tranquilizers, antidepressants, anesthetics, analgesics, anabolic steroids, estrogens, progesterones, glycosaminoglycans, polynucleotides, immunosuppressants and immunostimulants.

Also the above formulation can additionally include other additives up to 5% by weight. For instance, the composition can further comprise alcohol, polyol or Cremophor to improve the solubility of the insoluble drugs, tocopherol or tocopherol acetate to prevent oxidation, and fatty acid, fatty acid ester or fatty acid alcohol to increase drug absorption.

When applying these formulations in drug delivery system, it is preferred to use various administration routes including oral administration, buccal administration, mucosal administration, intranasal administration, intraperitoneal administration, subcutaneous injection, intramuscular injection, transdermal administration and intratumoral injection, and more preferably an oral administration.

The preparation method of the above formulation for the solubilization of insoluble drugs comprises the steps of:
1) solubilizing 4˜90% by weight of at least one monoglyceride compounds in 0.01˜90% by weight of at least one oil at temperatures lower than 50° C. to obtain a homogeneous mixture (step 1); and
2) solubilizing completely 0.01˜20% by weight of at least one insoluble drug in said mixture in step (1) (step 2).

The monoglycerides, oils and insoluble drugs used in preparing the solubilizing formulation are the same as described above.

In step (2) of the above preparation method, the said mixture can be stirred or sonicated in a bath type sonicator to speed up the solubilization process.

Also the above formulation can be prepared by the following method comprising the steps of:
1) mixing 4˜90% by weight of at least one monoglyceride compounds, 0.01˜90% by weight of at least one oil and 0.01˜20% of insoluble drug (step 1); and
2) preparing a homogeneous liquid by solubilizing said mixture in step (1) completely (step 2).

The monoglycerides, oils and insoluble drugs used in preparing the solubilizing formulation are the same as above.

In step (2) of the above preparation method, the said mixture can be stirred or sonicated in a bath type sonicator at temperatures lower than 50° C. to speed up the solubilization process.

The preparation methods described above are only examples of many possible methods, and other preparation method can also be used to obtain the above formulation for the solubilization of insoluble drug.

Also, the present invention provides the formulation for the solubilization of insoluble drug that uses the mucoadhesive composition including emulsifiers for solubilization of insoluble drugs as a drug delivery system.

More particularly, the above formulation is composed of 4˜90% by weight of at least one selected from the monoglycerides, 0.01˜90% by weight of at least one oil, 0.01˜90% by weight of at least one emulsifier and 0.01˜20% by weight of insoluble drug (with respect to the total weight of the composition).

The above formulation can be prepared by adding at least one monoglyceride, at least one oil, at least one emulsifier and insoluble drug at room or elevated temperatures.

The above monoglycerides are selected from a group consisting of one or more saturated or unsaturated monoglycerides having 10˜22 carbon atoms in the hydrocarbon chain. Monoglyceride is selected preferably from a group of consisting of monoolein, monopalmitolein, monomyristolein, monoelaidin and monoerucin, and semi-synthesized monoglycerides and their mixtures from triglycerides extracted from vegetable or animal oils, and more preferably monoolein.

The above oil solubilizing insoluble drugs is selected preferably from a group consisting of triglycerides, iodinated oil, vegetable oil or animal oil.

The above triglycerides are selected from a group consisting of one or more saturated or unsaturated triglycerides having 2˜20 carbon atoms in the hydrocarbon chain. For instance, triacetin, tributyrin, tricaproin, tricaprylin, tricaprin or triolein can be used.

The above iodized oils include iodized poppy seed oil such as Lipiodol, Ethiodol and iodized soybean oil.

The above vegetable oils include soybean oil, cottonseed oil, olive oil, poppyseed oil, linseed oil and sesame oil.

The above animal oils include squalane and squalene.

The above emulsifier is selected from the group consisting of phospholipid, a non-ionic surfactant, an anionic surfactant, a cationic surfactant, and bile acid.

The phospholipid is selected from the group consisting of a phosphatidylcholine (PC) and its derivative, a phosphatidylethanolamine (PE) and its derivative, a phosphatidylserine (PS) and its derivative and a polymeric lipid wherein a hydrophilic polymer is conjugated to the lipid headgroup.

The non-ionic surfactant is selected from the group consisting of a poloxamer (also known as Pluronic: polyoxyethylene-polyoxypropylene copolymer), a sorbitan ester (Span), a polyoxyethylene sorbitan (Tween) and a polyoxyethylene ether (Brij).

The anionic surfactant is selected from the group consisting of a phosphatidylserine (PS) and its derivative, a phosphatidic acid (PA) and its derivative and sodium dodecyl sulfate (SDS).

The cationic surfactant is selected from the group consisting of 1,2-dioleyl-3-trimethylammonium propane (DOTAP), dimethyldioctadecylammonium bromide (DDAB), N-[1-(1,2-dioleyloxy)propyl]-N,N,N-trimethylammonium chloride (DOTMA), 1,2-dioleyl-3-ethylphosphocholine (DOEPC) or 3β-[N-[(N′,N′-dimethylamino)ethan]carbamoyl]cholesterol (DC-Chol).

The bile acid is selected from the group consisting of cholic acid, its salt and derivatives; deoxycholic acid, its salt and derivatives; chenocholic acid, its salt and derivatives; and lithocholic acid, its salt and derivatives.

The above insoluble drugs that can be used in the present invention are antivirals, steroidal anti-inflammatory drugs (SAID), non-steroidal anti-inflammatory drugs (NSAID), antibiotics, antifungals, vitamins, hormones, retinoic acid, prostaglandins, prostacyclins, anticancer drugs, antimetabolitic drugs, miotics, cholinergics, adrenergic antagonists, anticonvulsants, antianxiety agents, major tranquilizers, antidepressants, anesthetics, analgesics, anabolic steroids, estrogens, progesterones, glycosaminoglycans, polynucleotides, immunosuppressants and immunostimulants

Other additives can be added to the above formulation including emulsifiers to be within 5% by weight. For instance, the composition can further comprise alcohol, polyol or Cremophor to improve the solubility of the insoluble drugs, tocopherol or tocopherol acetate to prevent oxidation, and fatty acid, fatty acid ester or fatty acid alcohol to increase drug absorption.

The formulations for solubilization of insoluble drugs with emulsifiers according to the present invention can be administered via various routes including oral administration, buccal administration, mucosal administration, nasal administration, intraperitoneal administration, subcutaneous injection, intramuscular injection, transdermal administration, intratumoral administration, and more preferably an oral administration.

The preparation method of the above formulation for the solubilization of insoluble drugs that uses the mucoadhesive composition including emulsifiers for solubilization of insoluble drugs as a drug delivery system comprises the steps of:
1) preparing a viscous liquid by solubilizing completely 4˜90% by weight of at least one monoglyceride compound, 0.01˜90% by weight of at least one oil and 0.01˜90% by weight of at least one emulsifier at temperatures lower than 50° C. to obtain a homogeneous mixture (step 1); and
2) preparing a homogeneous liquid formulation by mixing 0.01˜20% by weight of at least one insoluble drug in said liquid in step (1) (step 2).

The monoglycerides, oils, emulsifiers and insoluble drugs used in preparing the solubilizing formulation are the same as described above.

For example, after adding insoluble drug in a viscous liquid obtained by mixing completely monoglyceride compound, oil and emulsifier, the mixture can be stirred or sonicated for 3˜5 minutes at room temperature or temperatures lower than 50° C. to speed up the solubilization process.

Another preparation method of the above formulation for the solubilization of insoluble drugs that uses the mucoadhesive composition including emulsifiers for solubilization of insoluble drugs as a drug delivery system comprises the steps of:
1) preparing viscous liquid containing insoluble drug by mixing 0.01˜90% by weight of at least one oil and 0.01˜20% by weight of insoluble drug and sonicating in a bath type sonicator (step 1); and
2) preparing a homogeneous liquid formulation by mixing 0.01˜90% by weight of at least one emulsifier and 4˜90% by weight of at least one monoglyceride in said liquid in step (1) (step 2).

The monoglycerides, oils, emulsifiers and insoluble drugs used in preparing the solubilizing formulation are the same as above.

The preparation methods described above is only two of many possible methods, and other preparation methods can also be used to obtain the above formulation that uses the mucoadhesive composition including emulsifiers for solubilization of insoluble drugs as a drug delivery system.

The compositions for solubilization of insoluble drug of the present invention exist as liquid or in semi-solid state depending on the temperature at which they exist. The physical state of the formulation depends on the melting point. In general, the formulation exists as semi-solid at room temperature (ca. 25° C.), and as liquid at temperatures above room temperature. Also the melting point of the above formulation depends on the kinds and the amount of the additives. One of the general characteristics of the formulations is that they exist as a viscous liquid at body temperature and can be adsorbed on a wide area of the intestine.

The formulations of the present invention in viscous liquid, gel or semi-solid form are stable for a long period of time since the physical property of the composition does not change and the components including the insoluble drug do not degrade with time. Also the formulations for solubilization of insoluble drug of the present invention is an efficient solubilization system of the insoluble drugs since they can be easily dispersed in water or in aqueous solutions to produce particles bigger than 300 nm in diameter, and the dispersion does not form aggregates with time.

### BRIEF DESCRIPTION OF THE DRAWINGS

FIG. 1 is a graph showing the concentration of pyrene in blood and in different organs after oral administration of the liquid formulation solubilizing insoluble drug in Example 20 of the present invention. The quantitative analysis of pyrene was performed by HPLC. Tricaprylin emulsion including pyrene was orally administered as a control group.

-▪-; a group orally administered with liquid formulation for solubilization of insoluble drugs of the present invention (2 mg pyrene, weight ratio of the liquid formulation is monoolein:tricaprylin:pyrene=64.3:32.2:3.5, the liquid formulation in Example 20),

□-; a group orally administered with tricaprylin emulsion (2 mg pyrene, weight ratio of the emulsion is tricaprylin:tween 80:pyrene:water=8.65:0.95:0.4:90, the control group in Example 25).

FIG. 2 is a graph showing the concentration of pyrene in the intestine 1 or 2 hours after oral administration of the liquid formulation solubilizing insoluble drug in Example 20 of the present invention. The quantitative analysis of pyrene was performed by HPLC. Tricaprylin emulsion including pyrene was orally administered as a control group.

Test group: a group orally administered with liquid formulation for solubilization of insoluble drugs of the present invention (2 mg pyrene, weight ratio of the liquid formulation is monoolein:tricaprylin:pyrene=64.3:32.2:3.5, the liquid formulation in Example 20),

Control group: a group orally administered with tricaprylin emulsion (2 mg pyrene, weight ratio of the emulsion is tricaprylin:tween 80:pyrene:water=8.65:0.95:0.4:90, the control group in Example 25).

-▪-; pyrene concentration in the intestine 1 hour after oral administration,

-□-; pyrene concentration in the intestine 2 hours after oral administration.

FIG. 3 is a graph showing the concentration of pyrene in blood and in different organs after oral administration of the liquid formulation including emulsifier solubilizing insoluble drug in Example 24 of the present invention. The quantitative analysis of pyrene was performed by HPLC. Tricaprylin emulsion including pyrene was orally administered as a control group.

-▪-; a group orally administered with liquid formulation for solubilization of insoluble drugs of the present invention (2 mg pyrene, weight ratio of the liquid formulation is monoolein:tricaprylin:tween 80:pyrene=53.6:26.8:16.1:3.5, the liquid formulation in Example 24),

-□-; a group orally administered with tricaprylin emulsion (2 mg pyrene, weight ratio of the emulsion is tricaprylin:tween 80:pyrene:water 8.65:0.95:0.4:90, the control group in Example 25).

FIG. 4 is a graph showing the concentration of pyrene in the intestine 1 or 2 hours after oral administration of the liquid formulation including emulsifier solubilizing insoluble drug in Example 24 of the present invention. The quantitative analysis of pyrene was performed by HPLC. Tricaprylin emulsion including pyrene was orally administered as a control group.

Test group: a group orally administered with liquid formulation for solubilization of insoluble drugs of the present invention (2 mg pyrene, weight ratio of the liquid formulation is monoolein:tricaprylin:tween 80:pyrene=53.6:26.8:16.1:3.5, the liquid formulation in Example 24),

Control group: a group orally administered with tricaprylin emulsion (2 mg pyrene, weight ratio of the emulsion is tricaprylin:tween 80:pyrene:water=8.65:0.95:0.4:90, the control group in Example 25).

-▪-; pyrene concentration in the intestine 1 hour after oral administration,

-□-; pyrene concentration in the intestine 2 hours after oral administration.

### BEST MODE FOR CARRYING OUT THE INVENTION

This invention is explained in more detail based on the following Examples but they should not be construed as limiting the scope of this invention.

### EXAMPLE 1

### Mucoadhesive Composition for Solubilization of Insoluble Drugs Manufactured According to the Change in the Composition (1)

\textcircled{1} Manufacturing Mucoadhesive Composition for Solubilization of Insoluble Drugs

A mucoadhesive composition for solubilization of insoluble drugs, which is a viscous oily solution, was prepared by mixing 1 g monoolein and 0.5 g tricaprylin and warmed at 40° C. Monoolein used in Examples 1 and below was Myverol 18-99 K from Danisco A/S (Copenhagen, Denmark) with the monoolein content of 86.6 weight %.

\textcircled{2} Property Analysis of Thus Prepared Solubilizing Composition

The size of the emulsion particles were measured by using Malvern Zetasizer (Malvern Instruments Limited, England) after preparing the emulsion by adding 3 mL of distilled water to 2 μL of thus obtained liquid formulation. An average particle size and polydispersity were obtained by measuring values for a given formulation three times (Orr, Encyclopedia of emulsion technology, 1, 369-404, 1985). The polydispersity was obtained as the variance indicated by the logarithmic scale in the logarithmic normal distribution function. The above method in measuring the particle size and the polydispersity was used throughout the following examples.

The above composition exists as semi-solid or solid at room temperature and in a refrigerator, respectively, but as liquid at or above 40° C. Dispersion with the average particle size of 530 nm was obtained when the above composition was vortexed for 10 s in water. The absorbance at 400 nm was 2.36.

### EXAMPLE 2

### Mucoadhesive Composition for Solubilization of Insoluble Drugs Manufactured According to the Change in the Composition (2)

The composition was prepared by the same methods in Example 1 with the exception that 1 g monoolein and 1 g tricaprylin were used, and their particle size and polydispersity were measured by the same methods in Example 1. Dispersion with the average particle size of 730 nm was obtained. The absorbance at 400 nm was 2.23.

### EXAMPLE 3

### Mucoadhesive Composition for Solubilization of Insoluble Drugs Manufactured According to the Change in the Composition (3)

The composition was prepared by the same methods in Example 1 with the exception that 0.5 g monoolein and 1 g tricaprylin were used, and their particle size and polydispersity were measured by the same methods in Example 1. Dispersion with the average particle size of 554 nm was obtained. The absorbance at 400 nm was 2.54.

The results of the Examples 1-3 are summarized in the following Table 1.

TABLE 1

| Content (weight %) | | Particle size (nm) | Absorbance | |
|---|---|---|---|---|
| Monoolein | Tricaprylin | (polydispersity) | (400 nm) | Example |
| 66.7 | 33.3 | 530 (1) | 2.36 | 1 |
| 50 | 50 | 730 (1) | 2.23 | 2 |
| 33.3 | 66.7 | 554 (0.683) | 2.54 | 3 |

### COMPARATIVE EXAMPLE 1

Dispersion of Monoolein

Monoolein (99.5% purity) from Nu-Chek Prep (Elysian, Minn., USA) or Myverol 18-99K (monoolein content 86.6 weight %) from Danisco A/S (Copenhagen, Denmark) were mixed with water. Cubic phases were formed instead of dispersion. Since the cubic phase had very high viscosity and floated in water, the particle size or absorbance could not be determined.

### EXAMPLE 4

### Mucoadhesive Composition for Solubilization of Insoluble Drugs Manufactured According to the Change in the Oil and the Composition (1)

The composition was prepared by the same methods in Example 1 with the exception that 1 g monoolein and 0.5 g tributyrin were used. The particle size and polydispersity were measured by the same methods in Example 1. Dispersion with the average particle size of 303 nm was obtained. The absorbance at 400 nm was 0.78.

### EXAMPLE 5

### Mucoadhesive Composition for Solubilization of Insoluble Drugs Manufactured According to the Change in the Oil and the Composition (2)

The composition was prepared by the same methods in Example 1 with the exception that 1 g monoolein and 1 g tributyrin were used. The particle size and polydispersity were measured by the same methods in Example 1. Dispersion with the average particle size of 319 nm was obtained. The absorbance at 400 nm was 0.37.

### EXAMPLE 6

### Mucoadhesive Composition for Solubilization of Insoluble Drugs Manufactured According to the Change in the Oil and the Composition (3)

The composition was prepared by the same methods in Example 1 with the exception that 0.5 g monoolein and 1 g tributyrin were used. The particle size and polydispersity were measured by the same methods in Example 1. Dispersion with the average particle size of 916 nm was obtained. The absorbance at 400 nm was 2.19.

The results of the Examples 4-6 are summarized in the following Table 2.

TABLE 2

| Content (weight %) | | Particle size (nm) | Absorbance | |
|---|---|---|---|---|
| Monoolein | Tributyrin | (polydispersity) | (400 nm) | Example |
| 66.7 | 33.3 | 303 (0.246) | 0.78 | 4 |
| 50 | 50 | 319 (0.255) | 0.37 | 5 |
| 33.3 | 66.7 | 916 (1) | 2.19 | 6 |

### EXAMPLE 7

### Mucoadhesive Composition for Solubilization of Insoluble Drugs Manufactured According to the Change in the Oil (1)

The composition was prepared by the same methods in Example 1 with the exception that 1 g monoolein and 0.5 g squalane were used. The particle size and polydispersity were measured by the same methods in Example 1. An unstable dispersion with the average particle size of 1570 nm was obtained. The absorbance at 400 nm was 2.48.

### EXAMPLE 8

### Mucoadhesive Composition for Solubilization of Insoluble Drugs Manufactured According to the Change in the Oil (2)

The composition was prepared by the same methods in Example 1 with the exception that 1 g monoolein and 0.5 g lipiodol (Lipiodol Ultra-fluid, Laboratoire Guerbet, France, Iodine content: 38% by weight) were used. The particle size and polydispersity were measured by the same methods in Example 1. An unstable dispersion with the average particle size of 245 nm was obtained. The absorbance at 400 nm was 0.57.

The results of the Examples 7 and 8 are summarized in the following Table 3.

TABLE 3

| | Particle size (nm) | Absorbance | |
|---|---|---|---|
| Oil* | (polydispersity) | (400 nm) | Example |
| Squalane | 1570 (1.000) | 2.48 | 7 |
| Lipiodol | 245 (0.158) | 0.57 | 8 |

*Monoolein:Oil = 66.7:33.3 (Weight ratio)

### EXAMPLE 9

### Mucoadhesive Composition Including Emulsifiers for Solubilization of Insoluble Drugs Manufactured According to the Change in the Composition (1)

The composition was prepared by the same methods in Example 1 with the exception that 1 g monoolein, 0.5 g tricaprylin and 0.3 g Tween 80 were used. The particle size and polydispersity were measured by the same methods in Example 1. Dispersion with the average particle size of 583 nm was obtained. The absorbance at 400 nm was 2.68.

### EXAMPLE 10

### Mucoadhesive Composition Including Emulsifiers for Solubilization of Insoluble Drugs Manufactured According to the Change in the Composition (2)

The composition was prepared by the same methods in Example 1 with the exception that 1 g monoolein, 1 g tricaprylin and 0.3 g Tween 80 were used. The particle size and polydispersity were measured by the same methods in Example 1. Dispersion with the average particle size of 397 nm was obtained. The absorbance at 400 nm was 0.94.

### EXAMPLE 11

### Mucoadhesive Composition Including Emulsifiers for Solubilization of Insoluble Drugs Manufactured According to the Change in the Composition (3)

The composition was prepared by the same methods in Example 1 with the exception that 0.5 g monoolein, 1 g tricaprylin and 0.3 g Tween 80 were used. The particle size and polydispersity were measured by the same methods in Example 1. Dispersion with the average particle size of 587 nm was obtained. The absorbance at 400 nm was 1.32.

The results of the Examples 9-11 are summarized in the following Table 4.

TABLE 4

| Content (weight %) | | | Particle size | Absor- | |
|---|---|---|---|---|---|
| Mono- | Tri- | Tween | (nm) | bance | |
| olein | caprylin | 80 | (polydispersity) | (400 nm) | Example |
| 53.3 | 26.7 | 20 | 583 (1) | 2.68 | 9 |
| 40 | 40 | 20 | 397 (0.605) | 0.94 | 10 |
| 26.7 | 53.3 | 20 | 587 (0.211) | 1.32 | 11 |

### EXAMPLE 12

### Mucoadhesive Composition Including Emulsifiers for Solubilization of Insoluble Drugs Manufactured According to the Change in the Oil and the Composition (1)

The composition was prepared by the same methods in Example 1 with the exception that 1 g monoolein, 0.5 g tributyrin and 0.3 g Tween 80 were used. The particle size and polydispersity were measured by the same methods in Example 1. Dispersion with the average particle size of 1168 nm was obtained. The absorbance at 400 nm was 2.35.

### EXAMPLE 13

### Mucoadhesive Composition Including Emulsifiers for Solubilization of Insoluble Drugs Manufactured According to the Change in the Oil and the Composition (2)

The composition was prepared by the same methods in Example 1 with the exception that 1 g monoolein, 1 g tributyrin and 0.3 g Tween 80 were used. The particle size and polydispersity were measured by the same methods in Example 1. Dispersion with the average particle size of 170 nm was obtained. The absorbance at 400 nm was 0.41.

### EXAMPLE 14

### Mucoadhesive Composition Including Emulsifiers for Solubilization of Insoluble Drugs Manufactured According to the Change in the Oil and the Composition (3)

The composition was prepared by the same methods in Example 1 with the exception that 0.5 g monoolein, 1 g tributyrin and 0.3 g Tween 80 were used. The particle size and polydispersity were measured by the same methods in Example 1. Dispersion with the average particle size of 650 nm was obtained. The absorbance at 400 nm was 2.56.

The results of the Examples 12-14 are summarized in the following Table 5.

TABLE 5

| | | | | Absor- | |
|---|---|---|---|---|---|
| Content (weight %) | | | Particle size | bance | |
| Mono- | Tri- | Tween | (nm) | (400 nm) | |
| olein | butyrin | 80 | (polydispersity) | Monoolein | Example |
| 53.3 | 26.7 | 20 | 1168 (1) | 2.35 | 12 |
| 40 | 40 | 20 | 170 (0.946) | 0.41 | 13 |
| 26.7 | 53.3 | 20 | 650 (0.863) | 2.56 | 14 |

### EXAMPLE 15

### Mucoadhesive Composition Including Emulsifiers for Solubilization of Insoluble Drugs Manufactured According to the Change in the Oil (1)

The composition was prepared by the same methods in Example 1 with the exception that 1 g monoolein, 0.5 g squalane and 0.3 g Tween 80 were used. The particle size and polydispersity were measured by the same methods in Example 1. Dispersion with the average particle size of 506 nm was obtained. The absorbance at 400 nm was 1.75.

### EXAMPLE 16

### Mucoadhesive Composition Including Emulsifiers for Solubilization of Insoluble Drugs Manufactured According to the Change in the Oil (2)

The composition was prepared by the same methods in Example 1 with the exception that 1 g monoolein, 0.5 g lipiodol and 0.3 g Tween 80 were used. The particle size and polydispersity were measured by the same methods in Example 1. Dispersion with the average particle size of 913 nm was obtained. The absorbance at 400 nm was 3.10.

The results of the Examples 15-16 are summarized in the following Table 6.

TABLE 6

| | | Absorbance | |
|---|---|---|---|
| | Particle size (nm) | (400 nm) | |
| Oil* | (polydispersity) | Monoolein | Example |
| Squalane | 506 (0.407) | 1.75 | 15 |
| Lipiodol | 913 (0.472) | 3.10 | 16 |

*Monoolein:Oil:Tween 80 = 53.3:26.7:20 (Weight ratio)

### EXAMPLE 17

### Preparation of Mucoadhesive Formulation for Solubilization of Insoluble Drugs (1)

A mucoadhesive formulation for solubilization of insoluble drugs, which is a viscous oily solution, was prepared by mixing 1 g monoolein, 0.5 g tricaprylin and 15 mg cyclosporine A, an insoluble drug and warmed at 40° C., and their particle size and polydispersity were measured by the same methods in Example 1. Dispersion with the average particle size of 1525 nm was obtained. The absorbance at 400 nm was 1.39.

### EXAMPLE 18

### Preparation of Mucoadhesive Formulation for Solubilization of Insoluble Drugs (2)

A mucoadhesive formulation for solubilization of insoluble drugs was prepared by the same methods in Example 17 with the exception that 1 g monoolein, 0.5 g tricaprylin and 15 mg felodipin, an insoluble drug were used. The particle size and polydispersity were measured by the same methods in Example 1. Dispersion with the average particle size of 953 nm was obtained. The absorbance at 400 nm was 1.85.

The results of the Examples 17 and 18 are summarized in the following Table 7.

TABLE 7

| | Particle size (nm) | Absorbance | |
|---|---|---|---|
| Drug* | (polydispersity) | (400 nm) | Example |
| Cyclosporin A | 1525 (1) | 1.39 | 17 |
| Felodipin | 953 (1) | 1.85 | 18 |

*Monoolein:Tricaprylin:Drug = 66:33:1 (Weight ratio)

### EXAMPLE 19

### Preparation of Mucoadhesive Formulation for Solubilization of Insoluble Drugs (3)

Mucoadhesive formulations for solubilization of insoluble drugs were prepared by mixing 1 g of the composition prepared in Examples 1 through 8 and 0.4 mg pyrene and warmed at 40° C., and their particle size and polydispersity were measured by the same methods in Example 1.

The results of the Example 19 are summarized in the following Table 8.

TABLE 8

| | | | Content | Absorbance | Exam- |
|---|---|---|---|---|---|
| Content (weight %) | | | (weight %) | (400 nm) | ple |
| Monoolein | Tricaprylin | Pyrene | | | |
| 66.64 | 33.32 | 0.04 | 628 (1) | 2.19 | 1 |
| 49.98 | 49.98 | 0.04 | 729 (1) | 2.05 | 2 |
| 33.32 | 66.64 | 0.04 | 533 (1) | 2.48 | 3 |
| Monoolein | Tributyrin | Pyrene | | | |
| 66.64 | 33.32 | 0.04 | 503 (1) | 2.42 | 4 |
| 49.98 | 49.98 | 0.04 | 555 (1) | 2.47 | 5 |
| 33.32 | 66.64 | 0.04 | 698 (1) | 2.46 | 6 |
| Monoolein | Squalene | Pyrene | | | |
| 66.64 | 33.32 | 0.04 | 963 (1) | 2.70 | 7 |
| Monoolein | Lipiodol | Pyrene | | | |
| 66.64 | 33.32 | 0.04 | 246 (0.137) | 0.63 | 8 |

### EXAMPLE 20

### Preparation of Mucoadhesive Formulation for Solubilization of Insoluble Drugs (4)

A mucoadhesive formulation for solubilization of insoluble drugs was prepared by the same methods in Example 17 with the exception that 1 g monoolein, 0.5 g tricaprylin and 55 mg pyrene, an insoluble model drug, were used. The particle size and polydispersity were measured by the same methods in Example 1. Dispersion with the average particle size of 738 nm was obtained. The absorbance at 400 nm was 2.35.

### EXAMPLE 21

### Preparation of Mucoadhesive Formulation Including Emulsifiers for Solubilization of Insoluble Drugs (1)

A mucoadhesive formulation including emulsifiers for solubilization of insoluble drugs, which is a viscous oily solution, was prepared by mixing 1 g monoolein, 0.5 g tricaprylin, 0.3 mg Tween 80 and 18 mg cyclosporine A, an insoluble drug, were used. The particle size and polydispersity were measured by the same methods in Example 1. Dispersion with the average particle size of 1940 nm was obtained. The absorbance at 400 nm was 2.13.

### EXAMPLE 22

### Preparation of Mucoadhesive Formulation Including Emulsifiers for Solubilization of Insoluble Drugs (2)

A mucoadhesive formulation for solubilization of insoluble drugs was prepared by the same methods in Example 20 with the exception that 1 g monoolein, 0.5 g tricaprylin, 0.3 g Tween 80 and 18 mg felodipin, an insoluble drug, were used. The particle size and polydispersity were measured by the same methods in Example 1. Dispersion with the average particle size of 838 nm was obtained. The absorbance at 400 nm was 2.63.

The results of the Examples 21 and 22 are summarized in the following Table 9.

TABLE 9

| | | Absorbance | |
|---|---|---|---|
| | Particle size (nm) | (400 nm) | |
| Drug* | (polydispersity) | Monoolein | Example |
| Cyclosporin A | 1940 (1) | 2.13 | 21 |
| Felodipin | 838 (1) | 2.63 | 22 |

*Monoolein:Tricaprylin:Tween 80:Drug = 55:28:16:1 (Weight ratio)

### EXAMPLE 23

### Preparation of Mucoadhesive Formulation Including Emulsifiers for Solubilization of Insoluble Drugs (3)

Mucoadhesive formulations for solubilization of insoluble drugs was prepared by mixing 1 g of the composition prepared in Examples 9 through 16 and 0.4 mg pyrene and warmed at 40° C., and their particle size and polydispersity were measured by the same methods in Example 1.

The results of the Example 19 are summarized in the following Table 10.

TABLE 10

| | | | | Particle size | Absorbance | |
|---|---|---|---|---|---|---|
| | | | | (nm) | (400 nm) | |
| Content (weight %) | | | | (polydispersity) | Monoolein | Example |
| Monoolein | Tricaprylin | Tween 80 | Pyrene | | | |
| 53.31 | 26.66 | 19.99 | 0.04 | 668 (1) | 2.85 | 9 |
| 39.985 | 39.985 | 19.99 | 0.04 | 517 (1) | 2.74 | 10 |
| 26.66 | 53.31 | 19.99 | 0.04 | 764 (0.477) | 2.92 | 11 |
| Monoolein | Tributyrin | Tween 80 | Pyrene | | | |
| 53.31 | 26.66 | 19.99 | 0.04 | 721 (1) | 2.61 | 12 |
| 39.985 | 39.985 | 19.99 | 0.04 | 526 (1) | 2.89 | 13 |
| 26.66 | 53.31 | 19.99 | 0.04 | 588 (1) | 2.82 | 14 |
| Monoolein | Squalene | Tween 80 | Pyrene | | | |
| 53.31 | 26.66 | 19.99 | 0.04 | 400 (0.254) | 1.35 | 15 |
| Monoolein | Lipiodol | Tween 80 | Pyrene | | | |
| 53.31 | 26.66 | 19.99 | 0.04 | 643 (0.739) | 3.28 | 16 |

### EXAMPLE 24

### Preparation of Mucoadhesive Formulation Including Emulsifiers for Solubilization of Insoluble Drugs (4)

A mucoadhesive formulation for solubilization of insoluble drugs was prepared by the same methods in Example 20 with the exception that 1 g monoolein, 0.5 g tricaprylin, 0.3 g Tween 80 and 65.3 mg pyrene, an insoluble model drug, were used. The particle size and polydispersity were measured by the same methods in Example 1. Dispersion with the average particle size of 698 nm was obtained. The absorbance at 400 nm was 2.93.

### EXAMPLE 25

### In Vivo Oral Administration of Mucoadhesive Formulation for Solubilization of Insoluble Drugs

Animal experiments were performed by using the mucoadhesive formulations for the solubilization of insoluble drugs prepared in the above Example 20.

\textcircled{1} Oral Administration of Mucoadhesive Formulations for the Solubilization of Insoluble Drugs

Fifty-six microliters of the mucoadhesive formulation containing 2 mg pyrene was administered into Balb/C mouse (6˜7 weeks old, female) fasted for 4 hours previously by using a gastric sonde. Tricaprylin emulsion containing pyrene was prepared as a control group. Tricaprylin emulsion was prepared by mixing tricaprylin, tween 80 and pyrene at a weight ratio of 86.5:9.5:4 and solubilized completely by heating the mixture to 50° C. One milliliter of the mixture was mixed with 9 ml water and sonicated for 2 min by using a probe type sonicator (High intensity ultrasonic processor, microprocessor control, 600-Watt model). The particle size and the polydispersity of the prepared emulsion were 103 nm and 0.2, respectively, and the absorbance at 400 nm was 0.3. Tricaprylin emulsion (500 μl) containing 2 mg pyrene was administered orally for comparison. One, 2, 3, 4 and 6 h after the oral administration of the formulations, the concentrations of pyrene in the blood and in various organs were determined.

\textcircled{2} Determination of Pyrene Concentration in Blood and in Various Organs

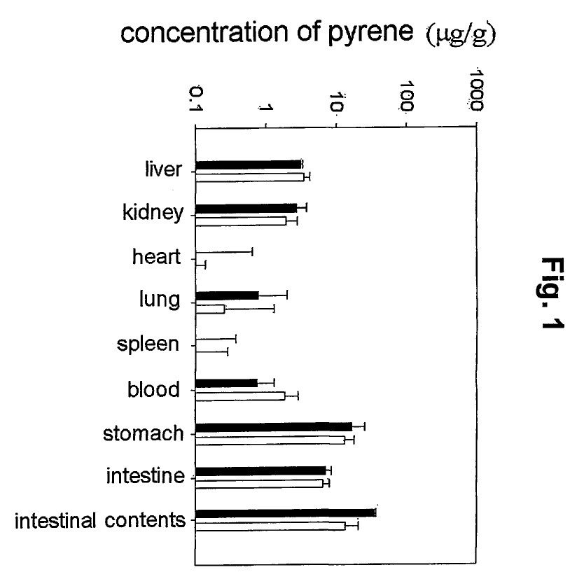

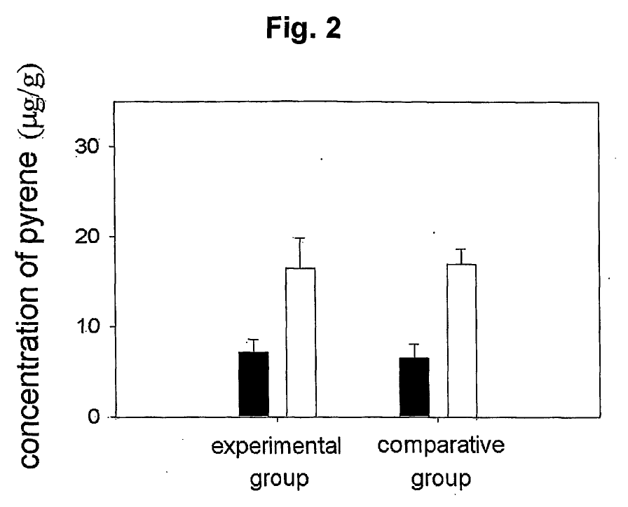

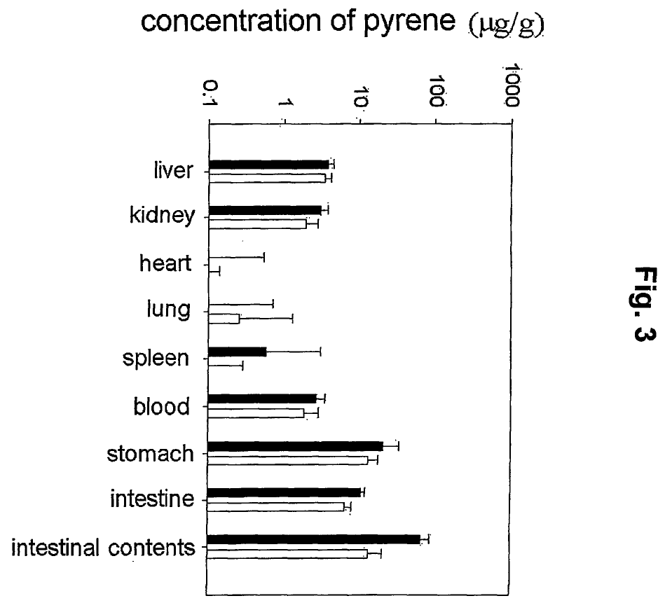

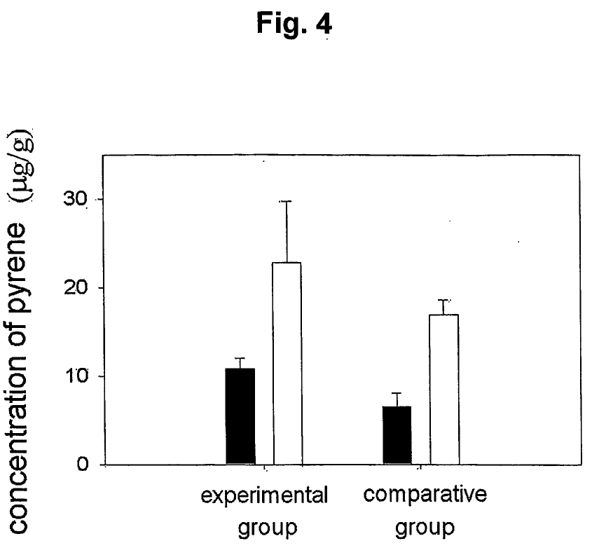

Blood withdrawn and organs taken out form the animal were mixed with methanol (8 folds in weight) and centrifuged at 14000 rpm at 4° C. for 15 min. After the mixture was centrifuged, the supernatant was taken to determine the concentration of pyrene by Fluorimetry (λₑₓ=336 nm, λₑₘ=389 nm). The concentrations of pyrene one hour after oral administration in each organ and in blood are shown in FIG. 1. It is well known that insoluble chemicals like pyrene can be absorbed into the intestinal cells when solubilized in hydrophobic particles, such as tricaprylin emulsion. It is notable that pyrene solubilized in the viscous liquid formulation of the present invention can be also absorbed into the body. Also the concentration of pyrene in the intestine increases with time similar to the case of tricaprylin emulsion control group as shown in FIG. 2.

### EXAMPLE 26

### In Vivo Oral Administration of Mucoadhesive Formulation Including Emulsifiers for Solubilization of Insoluble Drugs 1

Animal experiments were performed by using the mucoadhesive formulations for the solubilization of insoluble drugs prepared in the above Example 24.

\textcircled{1} Oral Administration of Mucoadhesive Formulations for the Solubilization of Insoluble Drugs

Fifty-six microliters of the mucoadhesive formulation containing 2 mg pyrene was administered into Balb/C mouse (6˜7 weeks old, female) fasted for 4 hours previously by using a gastric sonde. Tricaprylin emulsion containing pyrene was prepared and orally administered as a control group as in Example 25. One and two hours after the oral administration of the formulations, the concentrations of pyrene in the blood and in various organs were determined.

\textcircled{2} Determination of Pyrene Concentration in Blood and in Various Organs

The concentrations of pyrene one hour after oral administration in each organ and blood were quantified as in Example 25 and the result is shown in FIG. 3. It is well known that insoluble chemicals like pyrene can be absorbed into the intestinal cells when solubilized in hydrophobic particles, such as tricaprylin emulsion. It is notable that pyrene solubilized in a viscous liquid formulation of the present formulation can be absorbed into the body. Also the concentration of pyrene in the intestine increases with time higher than that in the case of tricaprylin emulsion control group as shown in FIG. 4.

### INDUSTRIAL APPLICABILITY

As described above, the mucoadhesive composition for solubilization of insoluble drugs according to the present invention can solubilize insoluble drug stably and also does not form precipitates of insoluble drug when dispersed in water. Since the mucoadhesive composition for solubilization of insoluble drugs according to the present invention can encapsulate and increase the absorption of insoluble drugs efficiently, it is suitable for oral and intraperitoneal delivery, and can be efficiently perish tumor cells.

1. A mucoadhesive composition for solubilization of insoluble drugs comprising 4˜90% by weight of at least one monoglyceride compound and 0.01˜90% by weight of at least one oil.

2. The mucoadhesive composition for solubilization of insoluble drugs according to claim 1, additionally comprising 0.01˜90% by weight of at least one emulsifier.

3. The mucoadhesive composition for solubilization of insoluble drugs according to claim 1, wherein said monoglyceride compound is chosen from a saturated or an unsaturated monoglyceride having 10˜22 carbon atoms in the hydrocarbon chain.

4. The mucoadhesive composition for solubilization of insoluble drugs according to claim 3, wherein said monoglyceride compound is chosen from monoolein, monopalmitolein, monomyristolein, monoelaidin, monoerucin, mixture of monoglycerides semi-synthesized from triglycerides of vegetable or animal oil.

5. The mucoadhesive composition for solubilization of insoluble drugs according to claim 1, wherein said oil is chosen from triglyceride, iodized oil, vegetable oil and animal oil.

6. The mucoadhesive composition for solubilization of insoluble drugs according to claim 5, wherein said triglyceride is chosen from saturated and unsaturated triglyceride having 2˜20 carbon atoms in each hydrocarbon chain.

7. The mucoadhesive composition for solubilization of insoluble drugs according to claim 5, wherein said triglyceride is chosen from triacetin, tributyrin, tricaproin, tricaprylin, tricaprin and triolein; wherein said iodized oil is chosen from Lipiodol, iodized poppy seed oil, Ethiodol and iodized soybean oil; wherein said vegetable oil is chosen from soybean oil, cottonseed oil, olive oil, poppyseed oil, linseed oil and sesame oil; and wherein said animal oil is chosen from squalane and squalene.

8.-10. (canceled)

11. The mucoadhesive composition for solubilization of insoluble drugs according to claim 2, wherein said emulsifier is chosen from a phospholipid, a non-ionic surfactant, an anionic surfactant, a cationic surfactant and a bile acid.

12. The mucoadhesive composition for solubilization of insoluble drugs according to claim 11, wherein said phospholipid is chosen from a phosphatidylcholine (PC) and its derivative, a phosphatidylethanolamine (PE) and its derivative, a phosphatidylserine (PS) and its derivative, and a polymeric lipid wherein a hydrophilic polymer is conjugated to the lipid headgroup; wherein said non-ionic surfactant is chosen from a poloxamer (Pluronic: polyoxyethylene-polyoxypropylene copolymer), a sorbitan ester (sorbitan esters; Span), a polyoxyethylene sorbitan (Tween) and a polyoxyethylene ether (Brij); wherein said anionic surfactant is chosen from a phosphatidylserine (PS) and its derivative, a phosphatidic acid (PA) and its derivative and sodium dodecyl sulfate (SDS); wherein said cationic surfactant is chosen from 1,2-dioleyl-3-trimethylammonium propane (DOTAP), dimethyldioctadecylammonium bromide (DDAB), N-[1-(1,2-dioleyloxy)propyl]-N,N,N-trimethylammonium chloride (DOTMA), 1,2-dioleyl-3-ethylphosphocholic acid (DOEPC) and 3β-[N-[(N′,N′-dimethylamino)ethan]carbamoyl]cholesterol (DC-Chol); and wherein said bile acid is chosen from cholic acid, its salt and derivatives, deoxycholic acid, its salt and derivatives; chenocholic acid, its salt and derivatives; and lithocholic acid, its salt and derivatives.

13.-16. (canceled)

17. The mucoadhesive composition for solubilization of insoluble drugs according to claim 1, additionally comprising 0.01˜5% by weight of another additive.

18. The mucoadhesive composition for solubilization of insoluble drugs according to claim 17, wherein the other additive is chosen from Cremophor, tocopherol, tocopherol acetate, fatty acids, fatty acid esters, fatty acid alcohols, alcohols and polyols.

19. The mucoadhesive composition for solubilization of insoluble drugs according to claim 18, wherein the other additive is chosen from an alcohol chosen from methanol, ethanol, propanol and isopropanol; and a polyol chosen from ethyleneglycol, propyleneglycol and polyethyleneglycol.

20. (canceled)

21. A preparation method of mucoadhesive composition for solubilization of insoluble drugs according to claim 1, wherein said method comprises the step of preparing a viscous liquid by solubilizing at least 4˜90% by weight of at least one monoglyceride compound in 0.01˜90% by weight of at least one oil.

22. The preparation method according to claim 21, wherein the said mixture is heated to 50° C. to speed up the solubilization process.

23. A preparation method of mucoadhesive composition for solubilization of insoluble drugs according to claim 2, wherein said method comprises the step of preparing a viscous liquid by mixing at least 4˜90% by weight of at least one monoglyceride compound and 0.01˜90% by weight of at least one oil with 0.01˜90% by weight of at least one emulsifier.

24. The preparation method according to claim 23 wherein the said mixture is heated to 50° C. to speed up the solubilization process.

25. The preparation method according to claim 23 wherein the said mixture is sonicated in a bath type sonicator to speed up the solubilization process.

26. A mucoadhesive formulation for solubilization of insoluble drugs comprising 4˜90% by weight of at least one monoglyceride compound, 0.01˜90% by weight of at least one oil and 0.01˜20% by weight of at least one insoluble drug.

27. The mucoadhesive formulation for solubilization of insoluble drugs according to claim 26, additionally containing 0.01˜90% by weight of at least one emulsifier.

28. The mucoadhesive formulation for solubilization of insoluble drugs according to claim 26, herein said monoglyceride compound is chosen from monoolein, monopalmitolein, monomyristolein, monoelaidin, monoerucin, and mixture of monoglycerides semi-synthesized from triglycerides of vegetable or animal oil.

29. The mucoadhesive formulation for solubilization of insoluble drugs according to claim 26, wherein said oil is chosen from triglyceride, iodized oil, vegetable oil and animal oil.

30. The mucoadhesive formulation for solubilization of insoluble drugs according to claim 29, wherein said triglyceride is chosen from saturated and unsaturated triglyceride having 2˜20 carbon atoms in each hydrocarbon chain.

31. The mucoadhesive formulation for solubilization of insoluble drugs according to claim 29, wherein said triglyceride is selected from a group consisting of triacetin, tributyrin, tricaproin, tricaprylin, tricaprin and triolein, wherein said iodized oil is chosen from Lipiodol, iodized poppy seed oil, Ethiodol and iodized soybean oil, wherein said vegetable oil is chosen from soybean oil, cottonseed oil, olive oil, poppyseed oil, linseed oil and sesame oil; and wherein said animal oil is chosen from squalane and squalene.

32.-34. (canceled)

35. The mucoadhesive formulation for solubilization of insoluble drugs according to claim 27, wherein said emulsifier is selected from a phospholipid, a non-ionic surfactant, an anionic surfactant, a cationic surfactant and a bile acid.

36. The mucoadhesive formulation for solubilization of insoluble drugs according to claim 35, wherein said phospholipid is chosen from a phosphatidylcholine (PC) and its derivative, a phosphatidylethanolamine (PE) and its derivative, a phosphatidylserine (PS) and its derivative and a polymeric lipid wherein a hydrophilic polymer is conjugated to the lipid headgroup; wherein said non-ionic surfactant is chosen from a poloxamer (Pluronic: polyoxyethylene-polyoxypropylene copolymer), a sorbitan ester (sorbitan esters; Span) a polyoxyethylene sorbitan (Tween) and a polyoxyethylene ether (Brij); wherein said anionic surfactant is chosen from a phosphatidylserine (PS) and its derivative, a phosphatidic acid (PA) and its derivative or sodium dodecyl sulfate (SDS); wherein said cationic surfactant is chosen from 1,2-dioleyl-3-trimethylammonium propane (DOTAP), dimethyldioctadecylammonium bromide (DDAB), N-[1-(1,2-dioleyloxy)propyl]-N,N,N-trimethylammonium chloride (DOTMA), 1,2-dioleyl-3-ethylphosphocholic acid (DOEPC) and 3β-[N-[(N′N′-dimethylamino)ethan]carbamoyl]cholesterol (DC-Chol); and wherein said bile acid is chosen from cholic acid, its salt and derivatives; deoxycholic acid, its salt and derivatives; chenocholic acid, its salt and derivatives; and lithocholic acid, its salt and derivatives.

37.-40. (canceled)

41. The mucoadhesive formulation for solubilization of insoluble drugs according to claim 26, wherein said bioactive compound is chosen from antivirals, steroidal anti-inflammatory drugs (SAID), non-steroidal anti-inflammatory drugs (NSAID), antibiotics, antifungals, vitamins, hormones, prostaglandins, prostacyclins, anticancer drugs, antimetabolitic drugs, miotics, cholinergics, adrenergic antagonists, anticonvulsants, antianxiety agents, major tranquilizers, antidepressants, anesthetics, analgesics, anabolic steroids, estrogens, progesterones, glycosaminoglycans, polynucleotides, immunosuppressants and immunostimulants.

42. The mucoadhesive formulation for solubilization of insoluble drugs according to claim 26, additionally comprising 0.01˜5% by weight of another additives.

43. The mucoadhesive formulation for solubilization of insoluble drugs according to claim 42, wherein the other additive is chosen from Cremophor, tocopherol, tocopherol acetate, fatty acids, fatty acid esters, fatty acid alcohols, alcohols and polyols.

44. The mucoadhesive formulation for solubilization of insoluble drugs according to claim 43, wherein the additive is chosen from an alcohol chosen from methanol, ethanol, propanol and isopropanol; and a polyol chosen from ethyleneglycol, propyleneglycol and polyethyleneglycol.

45. (canceled)

46. The mucoadhesive formulation for solubilization of insoluble drugs according to claim 26, wherein the administration route is chosen from oral administration, buccal administration, mucosal administration, intranasal administration, intraperitoneal administration, subcutaneous injection, intramuscular injection, transdermal administration and intratumoral injection.

47. The mucoadhesive formulation for solubilization of insoluble drugs according to claim 26 existing in liquid or semi-solid form.

48. A method of preparing the mucoadhesive formulation for solubilization of insoluble drugs according to claim 26, wherein said method comprises the steps of:
1) solubilizing 4˜90% by weight of at least one monoglyceride compound in 0.01˜90% by weight of at least one oil (step 1); and
2) solubilizing completely 0.01˜20% by weight of at least one insoluble drug in said mixture in step (1) by stirring (step 2).

49. The preparation method according to claim 48 wherein the said mixture is heated to 50° C. in step (1) to speed up the solubilization process.

50. The preparation method according to claim 48 wherein the said mixture is sonicated in a bath type sonicator in step (2) to speed up the solubilization process.

51. A preparation method of mucoadhesive formulation for solubilization of insoluble drugs according to claim 26, wherein said method comprises the step of preparing a homogenous liquid by mixing completely at least one monoglyceride compound, at least one oil and insoluble drug.

52. The preparation method according to claim 51 wherein the said mixture is heated to 50° C. and sonicated in a bath type sonicator to speed up the solubilization process.

53. A method of preparing the mucoadhesive formulation for solubilization of insoluble drugs according to claim 27, wherein said method comprises the steps of:
1) preparing a viscous liquid by mixing completely 4˜90% by weight of at least one monoglyceride compound, 0.01˜90% by weight of at least one oil and 0.01˜90% of at least one emulsifier (step 1); and
2) preparing a viscous liquid by mixing completely insoluble drug with said liquid in step (1) (step 2).

54. The preparation method according to claim 53 wherein the said liquid is heated to 50° C. in step (1) to speed up the solubilization process.

55. The preparation method according to claim 53 wherein the said liquid is heated to 50° C. in step (2) to speed up the solubilization process.

56. The preparation method according to claim 53 wherein the said liquid is sonicated in a bath type sonicator in step (2) to speed up the solubilization process.

57. A method of preparing the mucoadhesive formulation for solubilization of insoluble drugs according to claim 27, wherein said method comprises the steps of:
1) preparing oily liquid containing drug by solubilizing completely 0.01˜20% by weight of insoluble drug in 0.01˜90% by weight of at least one oil (step 1); and
2) preparing a homogeneous liquid by mixing completely said liquid in step (1) with 4˜90% by weight of at least one monoglyceride compound and 0.01˜90% of at least one emulsifier (step 2).

58. The preparation method according to claim 57, wherein the said liquid is heated to 50° C. and sonicated in a bath type sonicator in step (2) to speed up the solubilization process.
